(19) United States
(12) Patent Application Publication
(10) Pub. No.: US 2019/0096533 A1
(43) Pub. Date: Mar. 28, 2019

(54) METHOD AND SYSTEM FOR ASSISTIVE ELECTRONIC DETAILING ECOSYSTEM

(71) Applicant: Elements of Genius, Inc., Mt. Pleasant, SC (US)

(72) Inventor: Jonathan E. Ramaci, Mt. Pleasant, SC (US)

(21) Appl. No.: 16/144,506

(22) Filed: Sep. 27, 2018

Related U.S. Application Data
(60) Provisional application No. 62/564,707, filed on Sep. 28, 2017.

(51) Int. Cl. G16H 80/00; G10L 15/22; G16H 20/10; G16H 10/60

(52) U.S. Cl. CPC G16H 80/00; G10L 15/22; G10L 2015/225; G16H 10/60; G16H 20/10

(57) ABSTRACT

The present invention relates to computing devices, microcontrollers, memory storage devices, executable codes, methods, application software, automated voice recognition-response devices, natural language understanding-processing methods, algorithms, and IT communication system for electronic detailing (e-Detailing) and continuing medical education (CME). The system may be implemented in the form of a wearable device providing one or more features of voice, data, SMS reminders, and alerts. The device may function in combination with an application software accessible to multiple clients (users) executable on a remote server to provide education, support, and social contact for healthcare providers. Alternative embodiments implementing monitoring and intervention include using mobile apps or voice-controlled speech interface devices to access cloud control services capable of processing automated voice recognition-response and natural language understanding-processing to perform functions and fulfill user requests.

### CROSS-REFERENCE TO RELATED APPLICATIONS

The present application claims the benefit of U.S. Provisional Application 62/564,707, filed Sep. 28, 2017, and hereby incorporated by reference in its entirety.

### FIELD

The present disclosure relates to the field of medical information system; in particular, a method and system for an assistive electronic detailing ecosystem.

### BACKGROUND

Detailing can be defined as face-to-face (F2F) meetings/calls whereby pharmaceutical sales representatives (PSRs) communicate pharmaceutical/medical and marketing information to physicians. The global pharmaceutical industry utilizes detailing as the major marketing communication tool to facilitate interaction between physicians and PSRs. However, there are limitations with this traditional and most often used sales method and practice of information exchange. PSRs often wait a long time to meet with healthcare providers/doctors, and even once they meet with the doctor, are given only a short period of time. The efficiency of F2F detailing is dropping due to increased costs for PSRs, limitations in interactions between doctors and PSRs, and time constraints of doctors. The high costs of F2F detailing and sales promotion tools, inefficient sales efforts, biased and insufficient information about the drugs/medications, and physicians' limited time has decreased the attractiveness of F2F detailing. In response, due to the rapid development of information and communication technology, pharmaceutical companies have developed electronic detailing (e-Detailing).

E-Detailing is a technology-based solution for providing product information and promotional materials to physicians. e-Detailing systems vary in interactivity from those that provide relatively static product information online to those that require physicians to go through interactive product materials online. e-Detailing involves using digital technology, such as Internet, video conferencing, and interactive voice response, through which pharmaceutical companies target their marketing efforts toward specific physicians. These methods provide doctors with the latest information and knowledge at lower costs than F2F detailing, and they are intended to efficiently promote products to doctors to increase sales. Physicians can choose to engage in and learn about new pharmaceutical drug information at their own convenience. Pharmaceutical companies typically invite a chosen group of physicians to participate in an e-Detailing program. e-Detailing, with its flexibility and convenience, can be a relatively inexpensive and effective approach that can complement traditional detailing done by PSRs.

Virtual or interactive e-Detailing is a self-service product presentation that physicians can access in their own time. These presentations typically last between 5 and 15 minutes. The level of interaction can range from limited product information on handheld devices to more interactive web pages with incentive driven exercises. The appeal of such programs is that the physician is in control of its use. During a typical e-Detailing program, physicians are presented with a series of interactive learning exercises that reinforce messages specific to a pharmaceutical company's product. At the end of the exercise, physicians are asked whether they would like to receive samples, to meet a sales representative, to participate in market research surveys, or to request literature. Video e-Detailing is defined as F2F PC-based video conferencing between a physician and a pharmaceutical representative. Usually, physicians in this type of e-Detailing are provided with a preconfigured personal computer with all necessary applications preloaded and a webcam to see and speak with a PSR. The video image of the representative is displayed while audio communication is conducted over the telephone or microphone. Information about product indications, efficacy, dosage, side effects, and clinical data on new and existing products can also appear on the computer screen. In this type of e-Detailing, a physician can ask questions via a web interface. The PSR will guide the physician through the presentation, accompanying the online content with comments. Video e-Detailing is more closely related to traditional detailing than virtual e-Detailing.

Although e-Detailing facilitates interactive communication between a PSR and the physician, it has limitations. Even though most physicians use the Internet, some prefer offline drug information resources for convenience. Most physicians like to exploit F2F calls for information gathering and social interaction. Physicians deem PSRs as vital information sources, but physicians also report that detailing provides biased information and can compromise objectivity. The frequency of PSR calls may have a dual effect on the physicians' attitudes towards e-Detailing; too-frequent calls may be perceived as overwhelming considering time pressure and increased number of PSR interruptions during office hours. In such a situation, physicians' attitudes could be in favor of e-Detailing. Less-frequent PSR calls may give rise to negative attitudes towards e-Detailing, especially when physicians seek F2F interaction. Most physicians appreciate the personal touch that traditional detailing affords, and some choose not to write prescriptions for drugs unless they have a good relationship with the company that markets them. Pharmaceutical companies that seek to recapture detailing effectiveness must find solutions that address both the physicians' need for information and their time constraints, while enhancing the physician relationship.

Therefore, the need exists for a comprehensive solution to support the personal, social, interactive, efficient, and convenient delivery of medical information to healthcare providers. The ideal system should be integrated and incorporate an optimal infrastructure to support e-Detailing. The system should be provider-centered, comprehensive, coordinated, accessible 24/7, and committed to quality (e.g., accurate, non-biased, etc.). Such a system should enable an active and collaborative effort between healthcare providers (i.e. physician) and product manufacturers, in a mutually beneficial manner to improve the access, retrieval, and dissemination of medical-related (e.g., prescribing info, medical device, surgical procedures, clinical studies, medication, etc.) and marketing information.

Through applied effort, ingenuity, and innovation, Applicant has identified a number of deficiencies and problems with systems and methods for electronic detailing. Applicant has developed a solution that is embodied by the present invention, which is described in detail below.

### SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

In the broadest terms, the invention is an integrated assistive technology platform (system) incorporating one or more computing devices, microcontrollers, memory storage devices, executable codes, methods, software, automated voice recognition-response device, automated voice recognition methods, natural language understanding-processing methods, algorithms, and IT communication channels for electronic detailing (e-Detailing). The system incorporates an optimal infrastructure that is healthcare provider-centered, comprehensive, coordinated, accessible 24/7, and committed to quality. The system may incorporate the use of a wearable device providing one or more features of voice, data, SMS reminders, and alerts. The device may function in combination with an application software platform accessible to multiple clients (users) executable on one or more remote servers to provide healthcare providers (e.g., physician) support and medical information they value and trust; an e-Detailing ecosystem. The device may function in combination with one or more remote servers, cloud control services capable of providing automated voice recognition-response, natural language understanding-processing, applications for predictive algorithm processing, sending reminders, alerts, sending general and specific medical-related (e.g., disease, device, surgical procedures, clinical studies, medication, prescribing info, etc.) and marketing information. One or more components of the mentioned system may be implemented through an external system that incorporates a stand-alone speech interface device in communication with a remote server, providing cloud-based control service, to perform natural language or speech-based interaction with the user. The stand-alone speech interface device listens and interacts with a user to determine a user intent based on natural language understanding of the user's speech. The speech interface device is configured to capture user utterances and provide them to the control service. The control service performs speech recognition-response and natural language understanding-processing on the utterances to determine intents expressed by the utterances. In response to an identified intent, the controlled service causes a corresponding action to be performed. An action may be performed at the controlled service or by instructing the speech interface device to perform a function. The combination of the speech interface device and one or more applications executed by the control service serves as a relational agent. The relational agent provides conversational interactions, utilizing automated voice recognition-response, natural language processing, predictive algorithms, and the like, to perform functions, interact with the user (i.e. physician), fulfill user requests, and educate and inform the user.

In a preferred embodiment, the wearable device's form-factor is a hypoallergenic wrist watch, a wearable mobile phone, incorporating functional features that include, but are not limited to, voice, data, SMS text messaging, and alerts. In an alternative embodiment, the wearable device's form factor is an ergonomic and attachable-removable to-and-from an appendage or garment of a user as a pendant or the like. The wearable device may contain one or more of microprocessor, microcontroller, micro GSM/GPRS chipset, micro SIM module, read-only memory device, memory storage device, I-O devices, buttons, display, user interface, rechargeable battery, microphone, speaker, wireless transceiver, antenna, vibrating motor(output), preferably in combination, to function fully as a wearable mobile cellular phone. The said device enables communication with one or more remote servers capable of providing automated voice recognition-response, natural language understand-processing, predictive algorithm processing, reminders, alerts, general and specific information for e-Detailing. One or more components of the mentioned system may be implemented through an external system that incorporates a stand-alone speech interface device in communication with a remote server, providing cloud-based control service, to perform natural language or speech-based interaction with the user. The said device enables the user (i.e., physician, etc.) to interact with the said relational agent for accessing and retrieving medical-related information.

In another preferred embodiment, the wearable device can communicate with a secured remote server. The remote server is accessible through one or more computing devices, including but not limited to, desktop, laptop, tablet, mobile phone, smart appliances (i.e. smart TVs), and the like. The remote server contains an e-Detailing support application software that includes a database containing medical-related information. The application software provides a collaborative working environment to enable an active and collaborative effort between physicians and manufacturers/marketers to improve the access, retrieval, and dissemination of medical-related and marketing information. The software environment allows for, but is not limited to, sending-receiving text messages, sending-receiving voice messages, sending-receiving videos, streaming instructional videos, continuing medical education (CME) contents, or the like. The application software may interact with an electronic health or medical record system.

In an alternative embodiment, the said secured remote server is accessible using said stand-alone speech interface device or the speech interface is incorporated into one or more smart appliances, or mobile apps, capable of communicating with the same or another remote server, providing cloud-based control service, to perform natural language or speech-based interaction with the user, acting as said relational agent. The relational agent provides conversational interactions, utilizing automated voice recognition-response, natural language learning-processing, perform various functions and the like, to: interact with the user, fulfill user requests, educate, provide one or more skills, ask one or more questions, and store responses/answers.

In yet another embodiment, skills are developed and accessible through the relational agent. These skills may medical-related or marketing information that includes but is not limited to science, biology, chemistry, biochemistry, organic chemistry, molecular biology, pathology, scientific publications, clinical study results, adverse events, CME tests/contents, problem-based learning, practice guidelines, critical reading techniques, tutorials, medical procedures, prescribing info, medication errors, or the like.

In yet another embodiment, the user interacts with the relational agent via providing responses or answers to CME-related topics or problem-based learning curriculum. The questionnaires enable the assessment of the healthcare provider's proficiency or clinical competence. The responses or answers provided to the relational agent serve as input to one or more predictive algorithms to calculate a test score or provide certification. Such a profile can provide an assessment for the need of further continuing education.

Still further, an object of the present disclosure is an assistive electronic detailing system comprising a speech interface device operably engaged with a communications network, the speech interface device comprising a microphone, at least one non-transitory storage medium that stores instructions, and at least one processing unit that executes the instructions to receive a voice input from the microphone, process a voice transmission from the voice input, and communicate the voice transmission over the communications network according to at least one communications protocol, the voice transmission defining a user interaction; and, a remote server being operably engaged with the speech interface device via the communications network to receive the voice transmission, the remote server comprising at least one non-transitory storage medium storing instructions thereon, and at least one processing unit that executes the instructions to process the voice transmission and execute one or more actions in response to the voice transmission, the one or more actions comprising retrieving medical-related or detailing-related information from at least one database; communicating the medical-related or detailing-related information to the speech interface device; executing a communications protocol between the speech interface device and one or more third-party client devices to facilitate exchange of medical information between one or more users; and, storing data associated with the one or more actions in an application database.

Another object of the present disclosure is a method for access, retrieval, and dissemination of medical information comprising providing, with a computing device, a plurality of detailing information associated with one or more pharmaceutical products to a remote server; receiving, with a speech interface device, a voice input corresponding to a request for medical information; communicating, with the speech interface device via a communications network, the voice input to the remote server; processing, with the remote server executing an application software thereon, the voice input to execute one or more instructions associated with the application software; retrieving, with the remote server, medical-related or detailing-related information from at least one database according to the one or more instructions associated with the application software; and, communicating, with the remote server via the communications network, the medical-related or detailing-related information to the speech interface device.

Yet another object of the present disclosure is assistive electronic detailing system comprising a practitioner interface device operably engaged with a communications network, the practitioner interface device comprising a microphone, at least one non-transitory storage medium that stores instructions, and at least one processing unit that executes the instructions to receive a voice input from the microphone, process a voice transmission from the voice input, and communicate the voice transmission over the communications network according to at least one communications protocol, the voice transmission defining a user interaction; a remote server being operably engaged with the practitioner interface device via the communications network to receive the voice transmission, the remote server comprising at least one non-transitory storage medium storing instructions thereon, and at least one processing unit that executes the instructions to process the voice transmission and execute one or more actions in response to the voice transmission, the one or more actions comprising retrieving medical-related or detailing-related information from at least one database; communicating the medical-related or detailing-related information to the practitioner interface device; and, storing data associated with the one or more actions in an application database; and, a computing device associated with a pharmaceutical user, the computing device being operably engaged with the remote server via the communications network and configured to: provide a plurality of detailing-related information to the remote server; and, provide one or more communications to the practitioner interface device in response to a request by the remote server.

In summary, the integrated assistive technology platform enables the interactive, efficient, and convenient delivery of medical-related (e.g., disease, device, procedures, clinical studies, medication, prescribing info, etc.) information to healthcare providers. The system leverages a voice-controlled empathetic relational agent for e-Detailing. Such an ecosystem should enable an active and collaborative effort between healthcare providers (i.e. physician) and product manufacturers, in a mutually beneficial manner to improve the access, retrieval, and dissemination of medical-related and marketing information; assisting physicians to provide better care with access to information they value and trust.

The foregoing has outlined rather broadly the more pertinent and important features of the present invention so that the detailed description of the invention that follows may be better understood and so that the present contribution to the art can be more fully appreciated. Additional features of the invention will be described hereinafter which form the subject of the claims of the invention. It should be appreciated by those skilled in the art that the conception and the disclosed specific methods and structures may be readily utilized as a basis for modifying or designing other structures for carrying out the same purposes of the present invention. It should be realized by those skilled in the art that such equivalent structures do not depart from the spirit and scope of the invention as set forth in the appended claims.

### BRIEF DESCRIPTION OF DRAWINGS

The above and other objects, features and advantages of the present disclosure will be more apparent from the following detailed description taken in conjunction with the accompanying drawings, in which:

FIG. 1 is a system diagram of the integrated assistive technology system incorporating a portable mobile device, according to an embodiment of the present disclosure;

FIG. 2 is a diagram of the integrated assistive technology system incorporating a wearable mobile device, according to an embodiment of the present disclosure;

FIG. 3 is a perspective view of a wearable device and key features, according to an embodiment of the present disclosure;

FIG. 4 depicts an alternate wearing option and charging function, according to an embodiment of the present disclosure;

FIG. 5 is a graphical user interface containing the features of an application software platform providing an e-Detailing ecosystem for implementing the assistive technology platform, according to an embodiment of the present disclosure;

FIG. 6 is a diagram of the integrated assistive technology system incorporating a stand-alone voice-activated speech interface device, according to an embodiment of the present disclosure;

FIG. 7 illustrates the integrated assistive technology system incorporating a multimedia device, according to an embodiment of the present disclosure;

FIG. 8 is a functional block diagram of the elements of a relational agent, according to an embodiment of the present disclosure; and,

FIG. 9 is a process flow diagram of a method for access, retrieval, and dissemination of medical information, according to an embodiment of the present disclosure.

### DETAILED DESCRIPTION

Embodiments of the present invention will now be described more fully hereinafter with reference to the accompanying drawings, in which some, but not all, embodiments of the invention are shown. Indeed, the invention may be embodied in many different forms and should not be construed as limited to the embodiments set forth herein; rather, these embodiments are provided so that this disclosure will satisfy applicable legal requirements. Where possible, any terms expressed in the singular form herein are meant to also include the plural form and vice versa, unless explicitly stated otherwise. Moreover, certain terminology is used in the following description for convenience only and is not limiting. For example, as used herein, the term “a” and/or “an” shall mean “one or more,” even though the phrase “one or more” is also used herein. Furthermore, when it is said herein that something is “based on” something else, it may be based on one or more other things as well. In other words, unless expressly indicated otherwise, as used herein “based on” means “based at least in part on” or “based at least partially on.” The terminology includes the words above specifically mentioned, derivatives thereof, and words of similar import. Like numbers refer to like elements throughout.

This disclosure describes an integrated assistive technology platform for facilitating a high level of interaction between healthcare providers (e.g., physicians, nurses, etc.), peer-to-peer, and pharmaceutical/medical device manufacturers (herein referred to as “marketer”). The system leverages a voice-controlled empathetic relational agent for providing medical education, product support, medical affairs support, product information, access and retrieval of medical-related information, and social support. The platform enables the optimal access, retrieval, and dissemination of medical-related and marketing information; assisting physicians to provide better care with access to information they value and trust. In one embodiment, the platform or system comprises a combination of at least one of the following components: communication device; computing device; communication network; remote server; cloud server; cloud application software. The cloud server and service are commonly referred to as “on-demand computing”, “software as a service (SaaS)”, “platform computing”, “network-accessible platform”, “cloud services”, “data centers,” and the like. In an alternative embodiment, the intervention system comprises a combination of at least one voice-controlled speech interface device; computing device; communication network; remote server; cloud server; cloud application software. These components are configured to function together to enable a user to interact with a resulting relational agent. In addition, an application software, accessible by the user and others, using one or more remote computing devices, provides an environment, an e-Detailing ecosystem, to enable a voluntary, active, and collaborative effort between healthcare providers, peer-to-peer, or marketer in a mutually acceptable manner to improve communication and exchange of medical-related information.

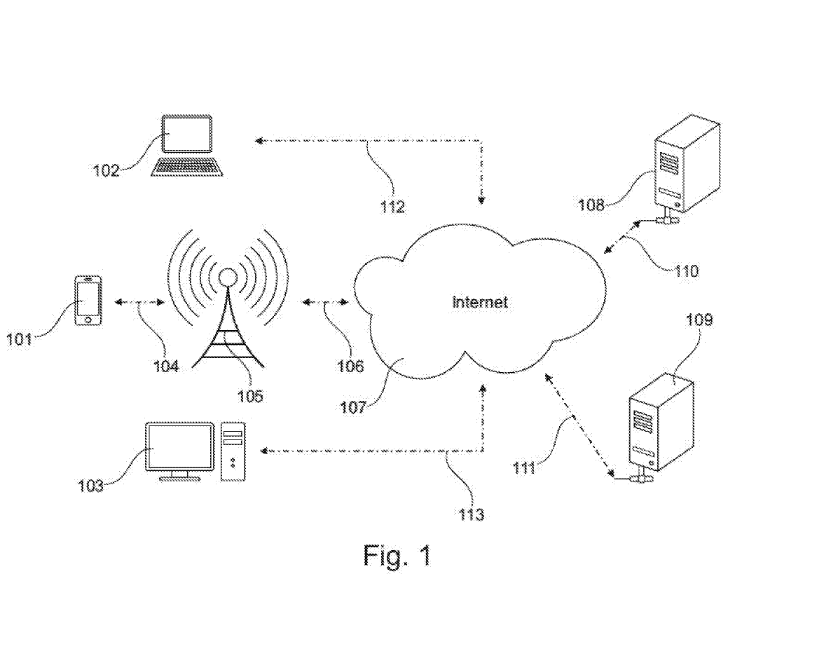

FIG. 1 illustrates the integrated assistive technology system incorporating a portable mobile device 101 for a healthcare provider to interact with one or more remote healthcare provider peer, or marketer. One or more user can access the system using a portable computing device 102 or stationary computing device 103. Device 101 communicates with the system via communication means 104 to one or more cellular communication network 105 which can connect device 101 via communication means 106 to the Internet 107. Device 101, 102, and 103 can access one or more remote servers 108, 109 via the Internet 107 through communication means 110 and 111 depending on the server. Device 102 and 103 can access one or more servers through communication means 112 and 113. Computing devices 101, 102, and 103 are preferable examples, but the computing devices may be any communication device, including tablet devices, cellular telephones, personal digital assistants (PDA), mobile Internet accessing devices, or other user systems including, but not limited to, pagers, televisions, gaming devices, laptop computers, desktop computers, cameras, video recorders, audio/video player, radio, GPS devices, any combination of the aforementioned, or the like. Communication means may comprise hardware, software, communication protocols, Internet protocols, methods, executable codes, instructions, known to one of ordinary skill in the art, and combined as to establish a communication channel between two or more devices. Communication means are available from one or more manufacturers. Exemplary communication means include wired technologies (e.g., wires, universal serial bus (USB), fiber optic cable, etc.), wireless technologies (e.g., radio frequencies (RF), cellular, mobile telephone networks, satellite, Bluetooth, etc.), or other connection technologies. The communications network employed to implement this invention may include of any type of communication network, including data and/or voice networks, and may be implemented using wired infrastructure (e.g., coaxial cable, fiber optic cable, etc.), a wireless infrastructure (e.g. RF, cellular, microwave, satellite, Bluetooth, etc.), and/or other connection technologies.

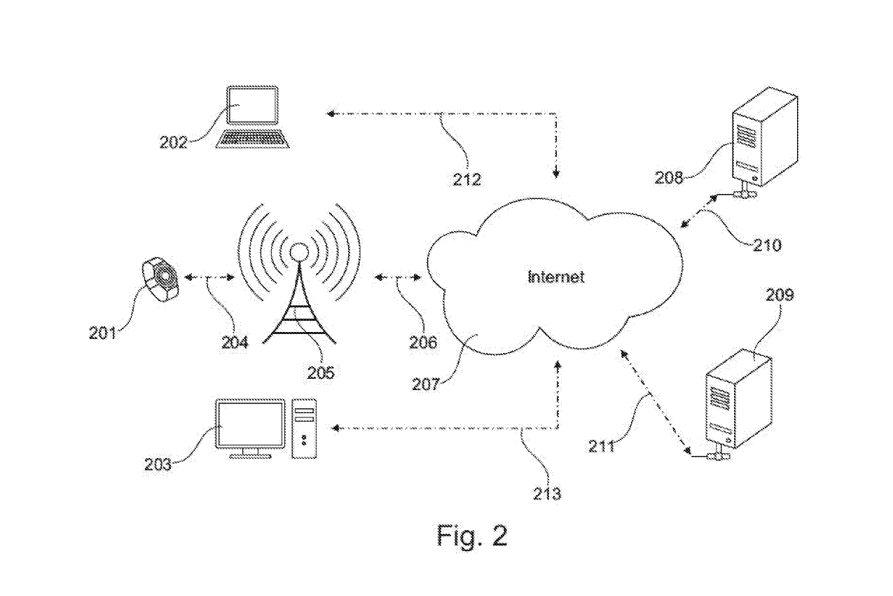

FIG. 2 illustrates the integrated assistive system incorporating a wearable device 201 for a healthcare provider to interact with one or more remote healthcare provider (e.g., a peer), or marketer. In a similar manner as illustrated in FIG. 1, one or more user can access the system using a portable computing device 202 or stationary computing device 203. Computing device 202 may be a laptop used by another healthcare provider or marketer. Stationary computing device 203 may reside, for example, at the facility of a marketer. Device 201 communicates with the system via communication means 204 to one or more cellular communication network 205 which can connect device 201 via communication means 206 to the Internet 207. Device 201, 202, and 203 can access one or more remote servers 208, 209 via the Internet 207 through communication means 210 and 211 depending on the server. Device 202 and 203 can access one or more servers through communication means 212 and 213.

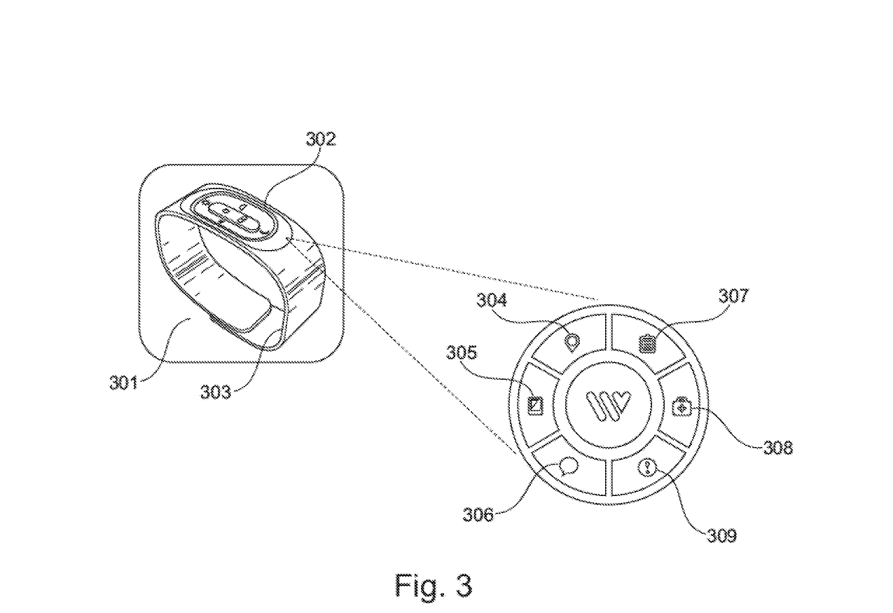

FIG. 3 is a pictorial rendering of the form-factor of a wearable device 301 (wrist watch) as a component of the integrated assistive technology system. The wearable device 301 is a fully functional mobile communication device (i.e., mobile cellular phone) that can be worn on the wrist of a user. The wearable device 301 comprises a watch-like device 302 snap-fitted onto a hypoallergenic wrist band 303. The watch-like device 302 provides a user-interface that allows a user to access features that include smart and secure location based services 304, mobile phone module 305, voice and data 306, advanced battery system and power management 307. The wearable device may contain one or more of microprocessor, microcontroller, micro GSM/GPRS chipset, micro SIM module, read-only memory device, memory storage device, I-O devices, buttons, display, user interface, rechargeable battery, microphone, speaker, wireless transceiver, antenna, accelerometer, vibrating motor, preferably in combination, to function fully as a wearable mobile cellular phone. A healthcare provider may use wearable device 301, depicted as device 201 of FIG. 2, to communicate with another peer or marketer. The wearable device 301 may allow a healthcare provider to access one or more remote cloud servers to communicate with a relational agent.

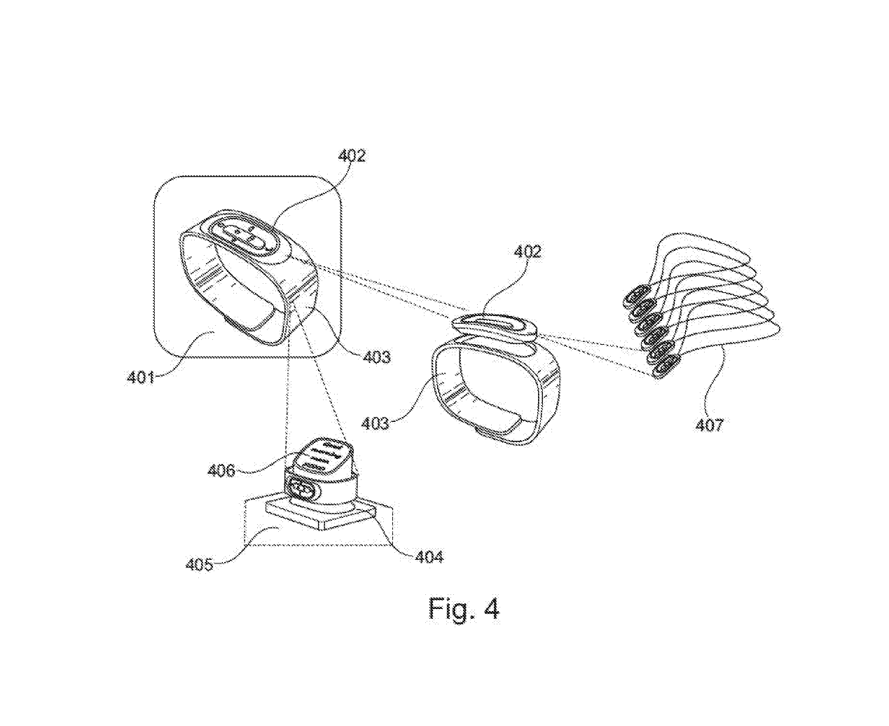

FIG. 4 illustrates details on additional features of the preferred wearable device. Wearable device 401 comprises a watch-like device 402 and wrist band 403, depicted in FIG. 3 as wearable device 301. Wearable device 401 can be stored together with a base station 404 and placed on top of platform 405. Platform 405 may be the surface of any furniture. Base station 404 contains electronic hardware, computing devices, and software to perform various functions, for example to enable the inductive charging of the rechargeable battery of wearable device 401, among others. Base station 404 also has a user interface 406 that can display visual information or provide voice messages to a user. Information can be in the form of greetings, reminders, phone messages, and the like. Watch-like device 402 is detachable from wrist band 403 and can be attached to band 407 to be worn by a user as a necklace.

The integrated assistive technology system of this invention utilizes an application software platform to create an e-Detailing ecosystem for provider medical education, product support, medical affairs support, product information dissemination, access and retrieval of medical-related information, and social support. The application software platform is stored in one or more servers 108, 109, 208, 209 as illustrated in FIG. 1, FIG. 2. The application software platform is accessible to users through one or more computing devices such as device 101, 102, 103, 201, 202, 203 described in this invention. Users of the application software can interact with each other via the said communication means. The software environment allows for, but is not limited to, sending-receiving text messages, sending-receiving voice messages, sending-receiving videos, streaming instructional videos, scheduling F2F detailing appointments, medical education information, CME, feedback to marketers, and the like. The application software can be used to store skills relating to science, biology, chemistry, biochemistry, organic chemistry, molecular biology, pathology, scientific publications, clinical study results, adverse events, CME tests/contents, problem-based learning, practice guidelines, critical reading techniques, tutorials, medical procedures, prescribing info, medication errors, or the like. The application software may interact with an electronic health or medical record system.

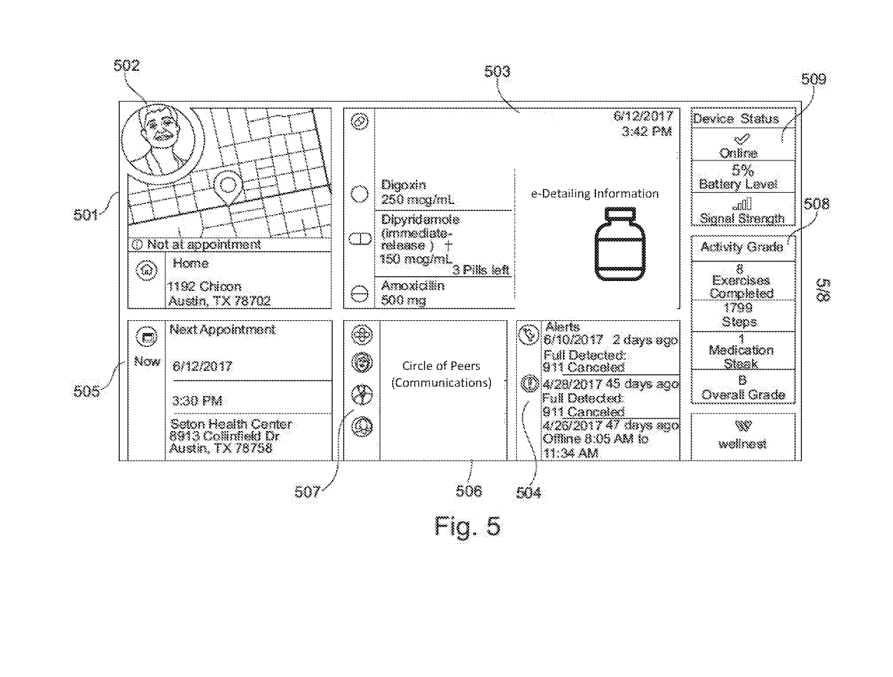

FIG. 5 is a screen-shot 501 that illustrates the type of information that users can generate using the application software platform. Screen-shot 501 provides an example of the information arranged in a specific manner and by no means limits the potential alternative or additional information that can be made available and displayed by the application software. In this example, a picture of healthcare provider 502 is presented at the upper left corner. The application may display the current location of the provider 502. A Medication Inventory 503 is available for review and contains a list of medications and related information. An Alerts 504 function is available to inform provider 502 of any new information (e.g., prescription label changes). A user (e.g., marketer, PSR) can review the F2F Next Appointment 505 information and schedule a visit to provider 502. A Circle of Peers 506 has pictures of the people 507 (e.g., other physicians, healthcare professionals) interacting with provider 502 in this e-Detailing ecosystem. A circle of peers enables information exchange among physicians, for example the latest clinical guidelines or patient success with a specific medication, etc. There's also an Activity Grade 508 that allows users (e.g., marketer) to monitor, for example, the activities/log-in of provider 502. Lastly, but not least, Device Status 509 provides information on the status of said wearable device, described for example in FIG. 3, as wearable device 301. The software application can be configurable, enabling specific said features and functions to be accessible, depending on user demographic, for example, a physician, a PSR, or marketer.

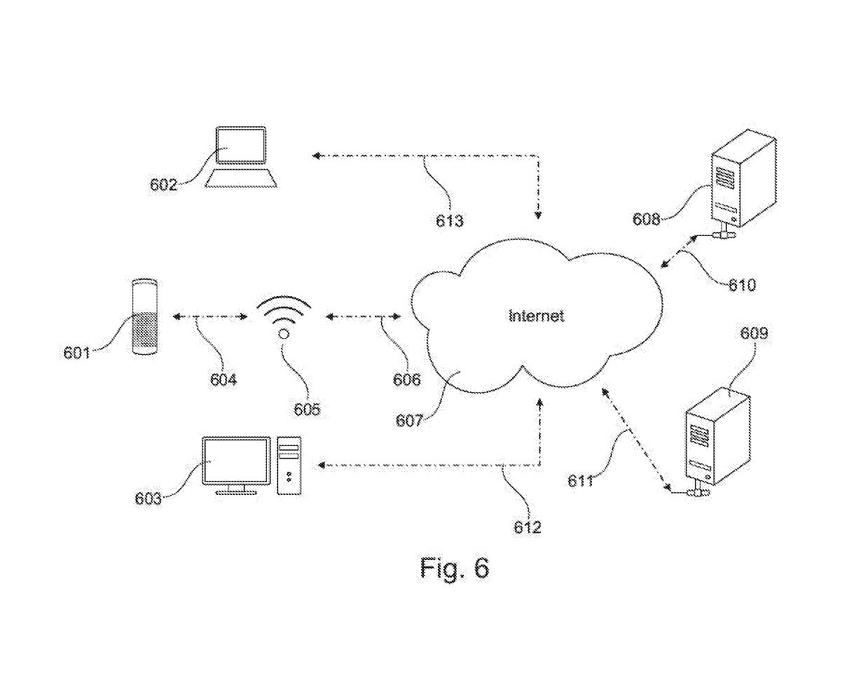

FIG. 6 illustrates the integrated assistive technology system incorporating a stand-alone voice-activated speech interface device 601 for a provider to interact with one or more remote user (e.g., peer, marketer) through a relational agent. In a similar manner as illustrated in FIG. 1, one or more user can access the system using a portable computing device 602 or stationary computing device 603. Computing device 602 may be a laptop used by another physician. Stationary computing device 603 may reside at the facility of a marketer. Device 601 communicates with the system via communication means 604 to one or more WiFi communication network 605 which can connect device 601 via communication means 606 to the Internet 607. Device 601, 602, and 603 can access one or more remote servers 608, 609 via the Internet 607 through communication means 610 and 611 depending on the server. Device 602 and 603 can access one or more servers through communication means 612 and 613. A user may request device 601 to call a marketer. Exemplary stand-alone speech interface devices include Echo, Dot, and Show; all available from Amazon (Seattle, Wash.).

In a preferred embodiment, the said stand-alone device 601 enables communication with one or more remote servers, for example server 608, capable of providing cloud-based control service, to perform natural language or speech-based interaction with the user. The stand-alone speech interface device 601 listens and interacts with a user to determine a user intent based on natural language understanding of the user's speech. The speech interface device 601 is configured to capture user utterances and provide them to the control service located on server 608. The control service performs speech recognition-response and natural language understanding-processing on the utterances to determine intents expressed by the utterances. In response to an identified intent, the controlled service causes a corresponding action to be performed. An action may be performed at the control service or by instructing the speech interface device 601 to perform a function. The combination of the speech interface device 601 and control service located on remote server 608 serves as a relational agent. The relational agent provides conversational interactions, utilizing automated voice recognition-response, natural language processing, predictive algorithms, and the like, to: perform functions, interact with the user, fulfill user requests, or educate the user. The relational agent may fulfill specific requests including calling a marketer or another healthcare provider. Ultimately the said device 601 enables the user to access and interact with the said relational agent to access and retrieve medical-related information, CME, education, product support, social contact support, feedback/communication for marketer, and the like. The information generated from the interaction of the user and the relational agent can be captured and stored in a remote server, for example remote server 609. This information may be incorporated into the application software as described in FIG. 5, making it accessible to multi-users (e.g., physicians, marketers, etc.) of the e-Detailing ecosystem of this invention.

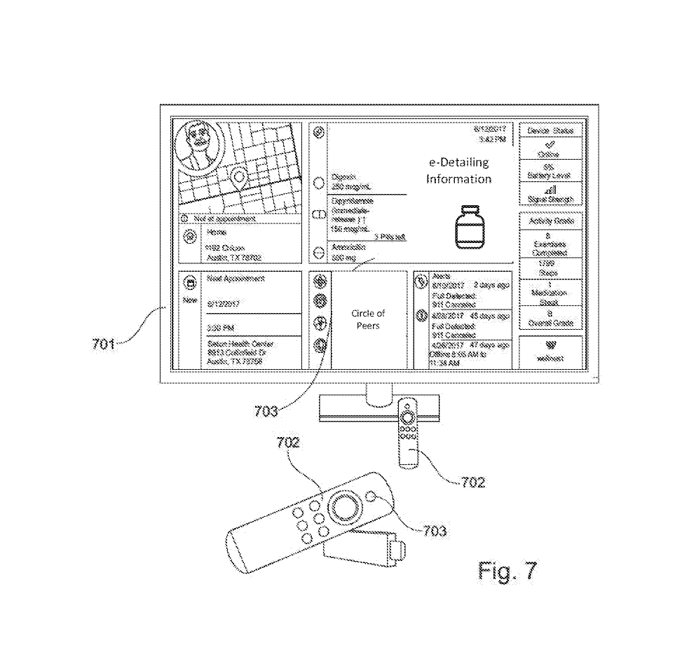

FIG. 7 illustrates the integrated assistive technology system incorporating a multimedia device 701 for a provider to interact with one or more remote peer provider or marketer through a relational agent. In a similar manner as illustrated in FIG. 6, one or more user can access the system using a remote-controlled device 702 containing a voice-controlled speech user interface 703. The multimedia device 701 is configured in a similar manner as device 601 of FIG. 6 as to enable a user to access application software platform depicted by screen-shot 704. The multimedia device 701 may be configured with hardware and software that enable streaming videos to be displayed. Exemplary products include FireTV, Fire HD8 Tablet, Echo Show; products available from Amazon.com (Seattle, Wash.), Nucleus (Nucleuslife.com), Triby (Invoxia.com), TCL Xcess, and the like. Streaming videos may include educational contents or materials for continuing medical education (CME), tutorials, podcasts, marketing materials, advertisements, etc. Preferable materials include contents and tools to increase provider knowledge and understanding of latest scientific discovery, drug discovery, industry news, clinical study results, clinical guidelines, adverse events, CME tests, problem-based learning contents, practice guidelines, critical reading techniques, tutorials, medical procedures, prescribing info, medication errors, regulatory announcements, or the like.

In an alternative embodiment, the function of the relational agent can be accessed through a mobile app and implemented through a system illustrated in FIG. 1. Such mobile app provide access to a remote server, for example remote server 108 of FIG. 1, capable of providing cloud-based control service, to perform natural language or speech-based interaction with the user. The mobile app contained in mobile device 101 monitors and captures voice commands and or utterances and transmits them through the said communication means to the control service located on server 108. The control service performs speech recognition-response and natural language understanding-processing on the utterances to determine intents expressed by the utterances. In response to an identified intent, the control service causes a corresponding action to be performed. An action may be performed at the control service or by responding to the user through the mobile app. The control service located on remote server 108 serves as a relational agent. The relational agent provides conversational interactions, utilizing automated voice recognition-response, natural language processing, predictive algorithms, and the like, to perform functions, interact with the user, and fulfill user requests. Ultimately the said device 101 enables the user to access and interact with the said relational agent for e-Detailing. The information generated from the interaction of the user and the relational agent can be captured and stored in a remote server, for example remote server 109. This information may be incorporated into the application software as described in FIG. 5, making it accessible to multi-users of the e-Detailing ecosystem of this invention.

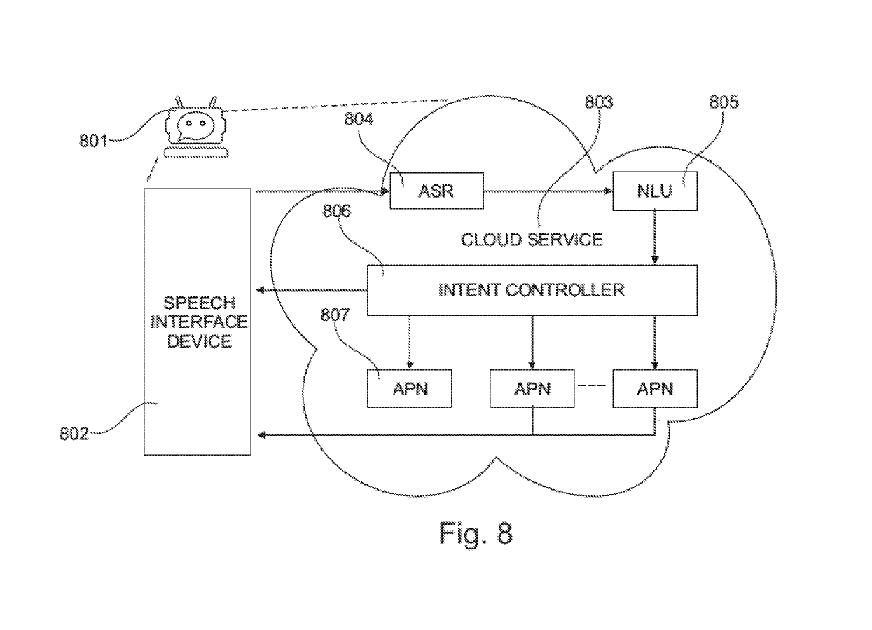

FIG. 8 illustrates a figurative relational agent 801 comprising the voice-controlled speech interface device 802 and a cloud-based control service 803. A representative cloud-based control service can be implemented through a SaaS model or the like. Model services include, but are not limited to, Amazon Web Services, Amazon Lex, Amazon Lambda, and the like, available through Amazon (Seattle, WA). Such a service provides access to one or more remote servers containing hardware and software to operate in conjunction with said voice-controlled speech interface device, app, or the like. Without being bound to a specific configuration, said control service may provide speech services implementing an automated speech recognition (ASR) function 804, a natural language understanding (NLU) function 805, an intent router/controller 806, and one or more applications 807 providing commands back to the voice-controlled speech interface device, app, or the like. The ASR function can recognize human speech in an audio signal transmitted by the voice-controlled speech interface device received from a built-in microphone. The NLU function can determine a user intent based on user speech that is recognized by the ASR components. The speech services may also include speech generation functionality that synthesizes speech audio. The control service may also provide a dialog management component configured to coordinate speech dialogs or interactions with the user in conjunction with the speech services. Speech dialogs may be used to determine the user intents using speech prompts. One or more applications can serve as a command interpreter that determines functions or commands corresponding to intents expressed by user speech. In certain instances, commands may correspond to functions that are to be performed by the voice-controlled speech interface device and the command interpreter may in those cases provide device commands or instructions to the voice-controlled speech interface device for implementing such functions. The command interpreter can implement “built-in” capabilities that are used in conjunction with the voice-controlled speech interface device. The control service may be configured to use a library of installable applications including one or more software applications or skill applications of this invention. The control service may interact with other network-based services (e.g., Amazon Lambda) to obtain information, access additional database, applications, or services on behalf of the user. A dialog management component is configured to coordinate dialogs or interactions with the user based on speech as recognized by the ASR component and or understood by the NLU component. The control service may also have a text-to-speech component responsive to the dialog management component to generate speech for playback on the voice-controlled speech interface device. These components may function based on models or rules, which may include acoustic models, specify grammar, lexicons, phrases, responses, and the like created through various training techniques. The dialog management component may utilize dialog models that specify logic for conducting dialogs with users. A dialog comprises an alternating sequence of natural language statements or utterances by the user and system generated speech or textual responses. The dialog models embody logic for creating responses based on received user statements to prompt the user for more detailed information of the intents or to obtain other information from the user. An application selection component or intent router identifies, selects, and/or invokes installed device applications and/or installed server applications in response to user intents identified by the NLU component. In response to a determined user intent, the intent router can identify one of the installed applications capable of servicing the user intent. The application can be called or invoked to satisfy the user intent or to conduct further dialog with the user to further refine the user intent. Each of the installed applications may have an intent specification that defines the serviceable intent. The control service uses the intent specifications to detect user utterances, expressions, or intents that correspond to the applications. An application intent specification may include NLU models for use by the natural language understanding component. In addition, one or more installed applications may contain specified dialog models that create and coordinate speech interactions with the user. The dialog models may be used by the dialog management component in conjunction with the dialog models to create and coordinate dialogs with the user and to determine user intent either before or during operation of the installed applications. The NLU component and the dialog management component may be configured to use the intent specifications of the applications either to conduct dialogs, to identify expressed intents of users, identify and use the intent specifications of installed applications, in conjunction with the NLU models and dialog modes, to determine when a user has expressed an intent that can be serviced by the application, and to conduct one or more dialogs with the user. As an example, in response to a user utterance, the control service may refer to the intent specifications of multiple applications, including both device applications and server applications, to identify, for example, a “drugs@FDA” intent. The service may then invoke the corresponding application. Upon invocation, the application may receive an indication of the determined intent and may conduct or coordinate further dialogs with the user to elicit further intent details. Upon determining sufficient details regarding the user intent, the application may perform its designed functionality in fulfillment of the intent. The voice-controlled speech interface device in combination with one or more functions 804,805,806 and applications 807 provided by the cloud service represents the relational agent 801 of the invention.

In a preferred embodiment, skills are developed for the relational agent 801 of FIG. 8 and stored as accessible applications within the cloud service 803. The skills contain information that enables the relational agent to respond to intents by performing an action in response to a natural language user input, information of utterances, spoken phrases that a user can use to invoke an intent, slots or input data required to fulfill an intent, and fulfillment mechanisms for the intent. These application skills may also reside in an alternative remote service, remote database (e.g., openFDA), the Internet, or the like, and yet be accessible to the cloud service 803. These skills may include but are not limited to intents for general topics, weather, news, music, pollen counts, flu events, UV conditions, adverse events, CME tests/contents, problem-based learning, practice guidelines, critical reading techniques, tutorials, medical procedures, prescribing info, medication errors, or the like. The skills enable the relational agent 801 to respond to intents and fulfill them through the voice-controlled speech interface device. These skills may be developed using application tools from vendors (i.e. Amazon Web Services, Alexa Skill Kits) providing cloud control services. The patient preferably interacts with relational agent 801 using skills to enable a voluntary, active, and collaborative effort between healthcare providers, peer-to-peer, or marketer in a mutually acceptable manner to improve communication and exchange of medical-related information.

It is a preferred object of this invention for relational agent 801 to provide access to one or more databases containing prescribing information (e.g., prescription drug package insert, etc.). These databases include, but are not limited to, drugs@FDA, orange book, First Databank, DailyMed, openFDA, proprietary database, or the like. Preferably, but not limited to, databases contain prescription information using Structure Product Labels. In an embodiment, a provider can query relational agent 801 for one or more of the following information of a specific approved drug: Indication and Usage, Dosage and Administration, Dosage Form and Strength, Contraindications, Adverse Reactions, Drug Interactions, Use in Specific Populations, Drug Description, Clinical Pharmacology, Toxicology, Storage and Handling Instructions, or the like.

It is a preferred object of this invention to utilize the spoken language interface as a natural means of interaction between the users and the system. Users can speak to the assistive technology similarly as they would normally speak to a human. It is understood, but not bound by theory, that verbal communication accompanied by the opportunity to engage in verbal conversation can improve communication and exchange of medical-related information. The relational agent may be used to engage providers in activities aimed at stimulating social functioning to leverage social support in lieu of F2F detailing. These skills may create a provider-centered environment that is responsive to the individual provider's preferences. The relational agent and one or more skills may be implemented in the engagement of a patient at an ambulatory setting (i.e., physician's office, clinic, etc.).

It is also an object of the present invention to provide a means to assess knowledge of specific diseases or conditions as part of continuing medication education (CME) programs. The responses-answers provided or obtained from questionnaires and instruments enable the assessment of provider proficiency. Upon a user intent, the relational agent can execute an algorithm or a pathway consisting of a series of questions that proceed in a state-machine manner, based upon yes or no responses, or specific response choices provided to the user. For example, a clinically validated structured multi-item, multidimensional, questionnaire scale may be used to assess knowledge of specific disease-state or clinical practice guidelines. The scale is preferably numerical, qualitative or quantitative, and allows for concurrent and predictive validity, with high internal consistency (i.e. high Cronbach's alpha), high sensitivity and specificity. Questions are asked by the relational agent and responses which may be in the form of yes/no answers from providers are recorded and processed by one or more skills. Responses may be assigned a numerical value, for example yes=1 and no=0. A high sum of yes in this case provides a measure of proficiency. One of ordinary skill in the art can appreciate the novelty and usefulness of using the relational agent of the present invention; a voice-controlled speech recognition and natural language processing combined with the utility of validated questionnaire scales. The questionnaire scales are constructed and implemented using skills developed through for example using the Alexa Skills Kit and or Amazon Lex. The combination of these modalities may be more conducive to eliciting information, providing feedback, and actively engaging providers during CME. The said scales may be modifiable with a variable number of items and may contain sub-scales with either yes/no answers, or response options, response options assigned to number values, Likert-response options, or Visual Analog Scale (VAS) responses. VAS responses may be displayed via a mobile app in the form of text messages employing emojis, digital images, icons, and the like.

In summary, the integrated assistive technology system of this invention enables a high level of interaction between healthcare providers, peers, PSRs, and marketers to improve the access, retrieval, and dissemination of medical-related and marketing information. The system leverages a voice-activated/controlled empathetic relational agent to enable the interactive, efficient, and convenient delivery of medical-related (e.g., disease, device, procedures, clinical studies, medication, prescribing info, etc.) information to healthcare providers. The system establishes an e-Detailing ecosystem that is provider-centered, comprehensive, coordinated, and accessible (24/7); enabling physicians to provide better care with access to medical-related information they value and trust. The system has utility for e-Detailing of pharmaceuticals and medical devices.

### Example 1

This example is intended to serve as a demonstration of the possible voice interactions between a relational agent and a patient with multimorbidity. The relational agent uses a control service (Amazon Lex) available from Amazon.com (Seattle, Wash.). Access to skills requires the use of a device wake word (“Alexa”) as well as an invocation phrase (“drugs@FDA”) for skills specifically developed for a proprietary wearable device that embodies one or more components of the present invention. The following highlights one or more contemplated capabilities and uses of the invention:

| Feature | Sample Phrases |
|---|---|
| Onboarding | “Alexa, open drugs@FDA” (conversation will continue) |
| Demo | |
| Checking | “Alexa, ask drugs@FDA if I have any messages” |
| Messages | “Alexa, tell drugs@FDA to check my messages” |
| Fire TV Video | “Alexa, ask drugs@FDA what is new on Fire TV about |
| Content | anti-hyperglycemic drugs” |
| e-Detailing | “Alexa, ask drugs@FDA to detail me about Lipitor” |
| | (conversation will continue) |
| | “Alexa, ask drugs@FDA the contraindication for |
| | Sitavig” (conversation will continue) |
| | “Alexa, call the Pfizer sales rep” |
| Reminders | “Alexa, ask drugs@FDA when my CME for herpes |
| | labialis is scheduled” |

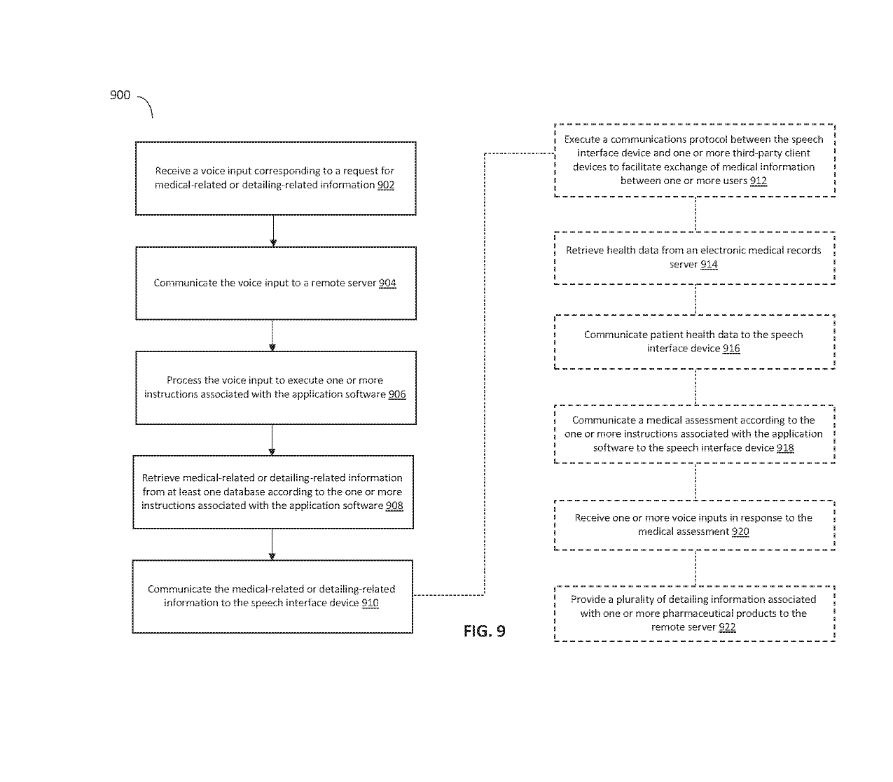

Referring now to FIG. 9, a process flow diagram of a method 900 for access, retrieval, and dissemination of medical information is shown. According to an embodiment of the present disclosure, method 900 may comprise receiving, with a speech interface device, a voice input corresponding to a request for medical-related or detailing-related information 902. According to certain embodiments, a computing device may provide a plurality of detailing information associated with one or more pharmaceutical products to the remote server 922. The speech interface device may then communicate, via a communications network, the voice input to a remote server 904. The remote server, executing an application software thereon, may process the voice input to execute one or more instructions associated with the application software 906. The remote server is then operable to receive medical-related or detailing-related information from at least one database according to the one or more instructions associated with the application software 908. The remote server, via the communications network, communicates the medical-related or detailing-related information to the speech interface device 910. According to an embodiment, the remote server, via the communications network, executes a communications protocol between the speech interface device and one or more third-party client devices to facilitate exchange of medical information between one or more users 912. The remote server, via an application programming interface, retrieves the patient health data from an electronic medical records server 914. In certain embodiments, the remote server, via the communications network, communicates the patient health data to the speech interface device 916. The remote server may be further operable to communicate a medical assessment according to the one or more instructions associated with the application software to the speech interface device 918. In response to receiving the medical assessment, the speech interface device may receive one or more voice inputs in response to the medical assessment 920.

While certain exemplary embodiments have been described and shown in the accompanying drawings, it is to be understood that such embodiments are merely illustrative of, and not restrictive on, the broad invention, and that this invention not be limited to the specific constructions and arrangements shown and described, since various other changes, combinations, omissions, modifications and substitutions, in addition to those set forth in the above paragraphs, are possible. Those skilled in the art will appreciate that various adaptations and modifications of the just described embodiments can be configured without departing from the scope and spirit of the invention. Therefore, it is to be understood that, within the scope of the appended claims, the invention may be practiced other than as specifically described herein.

What is claimed is:

1. An assistive electronic detailing system comprising:
a speech interface device operably engaged with a communications network, the speech interface device comprising a microphone, at least one non-transitory storage medium that stores instructions, and at least one processing unit that executes the instructions to receive a voice input from the microphone, process a voice transmission from the voice input, and communicate the voice transmission over the communications network according to at least one communications protocol, the voice transmission defining a user interaction; and,
a remote server being operably engaged with the speech interface device via the communications network to receive the voice transmission, the remote server comprising at least one non-transitory storage medium storing instructions thereon, and at least one processing unit that executes the instructions to process the voice transmission and execute one or more actions in response to the voice transmission, the one or more actions comprising:
retrieving medical-related or detailing-related information from at least one database;
communicating the medical-related or detailing-related information to the speech interface device;
executing a communications protocol between the speech interface device and one or more third-party client devices to facilitate exchange of medical information between one or more users; and,
storing data associated with the one or more actions in an application database.

2. The system of claim 1 wherein the speech interface device is configured as a wearable device.

3. The system of claim 1 wherein the one or more actions further comprise retrieving the medical-related or detailing-related information from at least one third-party database via an application programming interface.

4. The system of claim 1 wherein the one or more actions further comprise communicating a conversational interaction to the speech interface device.

5. The system of claim 4 wherein the conversational interaction comprises a continuing medical education assessment.

6. The system of claim 1 wherein the remote server is operably engaged with an electronic medical records server via an application programming interface.

7. The system of claim 6 wherein the one or more actions further comprise communicating patient-specific medical information to the speech interface device.

8. The system of claim 6 wherein the one or more actions further comprise communicating patient-specific detailing information to the speech interface device.

9. A method for access, retrieval, and dissemination of medical information comprising:
receiving, with a speech interface device, a voice input corresponding to a request for medical-related or detailing-related information;
communicating, with the speech interface device via a communications network, the voice input to a remote server;
processing, with the remote server executing an application software thereon, the voice input to execute one or more instructions associated with the application software;
retrieving, with the remote server, medical-related or detailing-related information from at least one database according to the one or more instructions associated with the application software; and,
communicating, with the remote server via the communications network, the medical-related or detailing-related information to the speech interface device.

10. The method of claim 9 further comprising executing, with the remote server via the communications network, a communications protocol between the speech interface device and one or more third-party client devices to facilitate exchange of medical information between one or more users.

11. The method of claim 9 further comprising retrieving, with the remote server via an application programming interface, patient health data from an electronic medical records server.

12. The method of claim 11 further comprising communicating, with the remote server via the communications network, the patient health data to the speech interface device.

13. The method of claim 9 further comprising communicating, with the remote server, a medical assessment according to the one or more instructions associated with the application software to the speech interface device.

14. The method of claim 13 further comprising receiving, with the speech interface device, one or more voice inputs in response to the medical assessment.

15. The method of claim 9 further comprising providing, with a computing device, a plurality of detailing information associated with one or more pharmaceutical products to the remote server.

16. An assistive electronic detailing system comprising:
a practitioner interface device operably engaged with a communications network, the practitioner interface device comprising a microphone, at least one non-transitory storage medium that stores instructions, and at least one processing unit that executes the instructions to receive a voice input from the microphone, process a voice transmission from the voice input, and communicate the voice transmission over the communications network according to at least one communications protocol, the voice transmission defining a user interaction;
a remote server being operably engaged with the practitioner interface device via the communications network to receive the voice transmission, the remote server comprising at least one non-transitory storage medium storing instructions thereon, and at least one processing unit that executes the instructions to process the voice transmission and execute one or more actions in response to the voice transmission, the one or more actions comprising:
retrieving medical-related or detailing-related information from at least one database;
communicating the medical-related or detailing-related information to the practitioner interface device; and,
storing data associated with the one or more actions in an application database; and,
a computing device associated with a pharmaceutical user, the computing device being operably engaged with the remote server via the communications network and configured to:
provide a plurality of detailing-related information to the remote server; and,
provide one or more communications to the practitioner interface device in response to a request by the remote server.

17. The system of claim 16 wherein the practitioner interface device is configured as a wearable device.

18. The system of claim 16 wherein the remote server is operably engaged with an electronic medical records server via an application programming interface.

19. The system of claim 18 wherein the one or more actions further comprise communicating patient-specific medical information to the practitioner interface device.

20. The system of claim 18 wherein the one or more actions further comprise communicating patient-specific detailing information to the practitioner interface device.
